(19) United States
(12) Patent Application Publication
(10) Pub. No.: US 2015/0000023 A1
(43) Pub. Date: Jan. 1, 2015

(54) URINARY AID

(71) Applicant: Heather Massey, Mooresville, NC (US)

(72) Inventor: Heather Massey, Mooresville, NC (US)

(21) Appl. No.: 13/927,482

(22) Filed: Jun. 26, 2013

(51) Int. Cl. A61F 5/453; A61F 5/44

(52) U.S. Cl. CPC A61F 5/453; A61F 5/4404; A61F 2005/4402
USPC 4/301

(57) ABSTRACT

A urinal aid comprising: a funnel shaped device, a flexible conduit having a proximal end connected to the funnel shaped device and a distal end directed toward a toilet bowl, and a toilet engagement device detachably engaged with the flexible conduit. The toilet engagement device is selected from either a clamping means detachably engaged with the flexible conduit and a bowl hook detachably engaged to the clamping means where the hook is engaged to a toilet bowl to secure the urinary aid to the toilet bowl and hold the urinary aid in place or an arm bracket detachably engaged with the flexible conduit where the arm bracket is engaged to a toilet bowl to secure the urinary aid to the side of the toilet bowl and hold the urinary aid in place.

### FIELD OF THE INVENTION

The present invention relates to a device used as a urination aid.

### BACKGROUND OF THE INVENTION

Any parent can tell you that when it comes to urination, males do not always have the best aim. This is especially true for young boys just learning how to use a toilet. The result is a frequent need to wipe down and clean the rim, outer bowl and area surrounding the toilet. There are several devices which have been presented in the past in order to help remedy this problem. However, most of those devices are rather complex and may even require permanent attachment to a toilet. Examples of these devices include:

U.S. Pat. No. 3,500,480 which discloses a urinal device which includes a pivoting arm mounted to the tank of a toilet which holds a urinal device in place while in use. This device also utilized a very large and cumbersome sleeve to direct the urine into the toilet which makes removing the device very difficult.

U.S. Pat. No. 3,822,419 which discloses a urinal device which is permanently secured to the underside of a toilet seat and requires a user to stand laterally of the toilet seat in order to use the device. The permanent attachment would make cleaning this device rather difficult.

U.S. Pat. No. 4,612,676 which discloses a urinal device which includes a pivoting arm mounted behind the seat, next to the toilet tank, which hold a urinal device in place while in use. The device then swings up and stores the device above the toilet tank, looking very unsightly.

U.S. Pat. No. 4,612,676 which discloses a device which sits right on top of the toilet rim, making the height of the device very low and difficult for anyone taller than about 36 inches. The lack of any kind of conduit or extension vastly limits the practicality of this device.

U.S. Pat. No. 5,737,779 which discloses a urinal device which is permanently affixed to both the bowl and the tank of a toilet. The device includes a funnel and a discharge fluid flush assembly and must be held by the user while in use. This design is simply not practical for a potty training child to use as just urinating is enough of a task for a 2-4 year old let alone having to “aim” and hold a device up.

U.S. Pat. No. 6,154,891 which discloses a device which must be held up by a string when in use and simply dangles over the toilet bowl. Again, this design is simply not practical for a potty training child to use as just urinating is enough of a task for a 2-4 year old let alone having to “aim” and hold a device up by a string.

In light of all of the inadequacies with existing urinal aids detailed above, it is clear that a need exists to provide a device which aids a person with urination which is both hygienic and simple enough for a child to use.

### SUMMARY OF THE INVENTION

A urinal aid comprising: a funnel shaped device, a flexible conduit having a proximal end connected to the funnel shaped device and a distal end directed toward a toilet bowl, and a toilet engagement device detachably engaged with the flexible conduit. The toilet engagement device is selected from either a clamping means detachably engaged with the flexible conduit and a bowl hook detachably engaged to the clamping means where the hook is engaged to a toilet bowl to secure the urinary aid to the toilet bowl and hold the urinary aid in place or an arm bracket detachably engaged with the flexible conduit where the arm bracket is engaged to a toilet bowl to secure the urinary aid to the side of the toilet bowl and hold the urinary aid in place.

### BRIEF DESCRIPTION OF THE DRAWINGS

FIG. 1 is a side view of the urinal aid.

FIG. 2 is a proximal view of the urinal aid.

FIG. 3 is a proximal view of the urinal aid.

FIG. 4 is a front view of a urinal aid engaged to a toilet.

FIG. 5 is a proximal view of a urinal aid engaged to a toilet.

FIG. 6 is a side view of the urinal aid.

FIG. 7 is a top and side view of an arm bracket.

FIG. 8 is a proximal view of a urinal aid engaged to a toilet.

FIG. 9 is a side view of the urinal aid.

FIG. 10 is a top and side view of an arm bracket.

FIG. 11 is a proximal view of a urinal aid engaged to a toilet.

### DETAILED DESCRIPTION

The instant invention describes a urinal aid 10 which is a device designed to assist an individual with the act of urination. Generally, a urinal aid is designed for use by young males, but the device may be adapted for use by males of any age. Looking to the figures one can see that the instant invention describes a urinal aid 10 which is comprised of a funnel shaped device 20 which has a wide opening 22 at its proximal end 26 and a narrow opening 24 at its distal end 28. A flexible conduit 30 which has a proximal end 36 and a distal end 38 is engaged to the funnel shaped device 20. The proximal end 36 of the flexible conduit 30 is engaged to the distal end 28 of the funnel shaped device 20 and the distal end 38 of the flexible conduit is directed toward a toilet bowl 44. A clamping means 60 is detachably engaged with the flexible conduit 30 and a bowl hook 70 is detachably engaged to the clamping means 70. The bowl hook 70 is engaged to a toilet bowl 44 to secure the urinary aid 10 to the side of the toilet bowl 44 and hold the urinary aid 10 in place and may be rapidly detached from the toilet bowl 44.

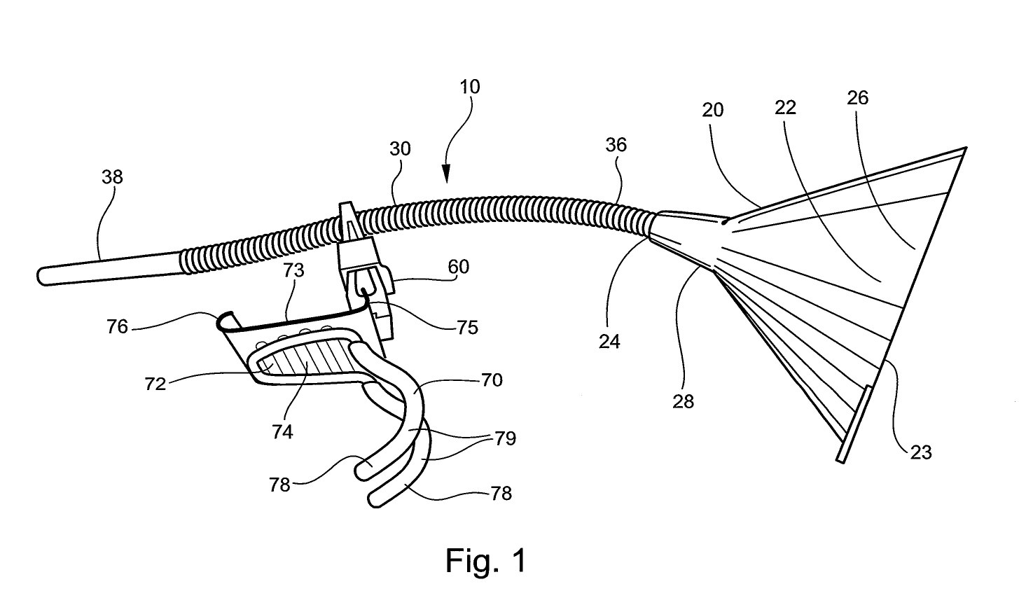

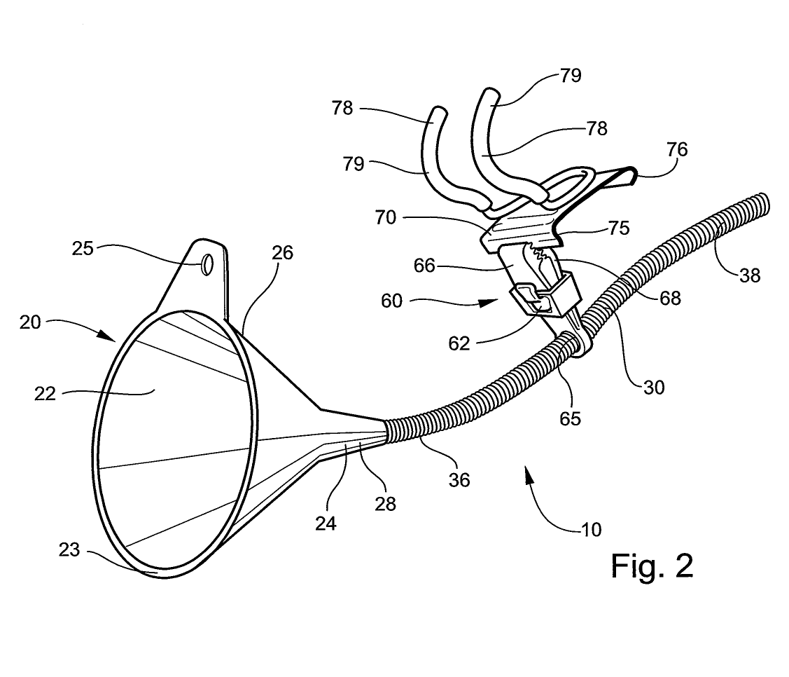

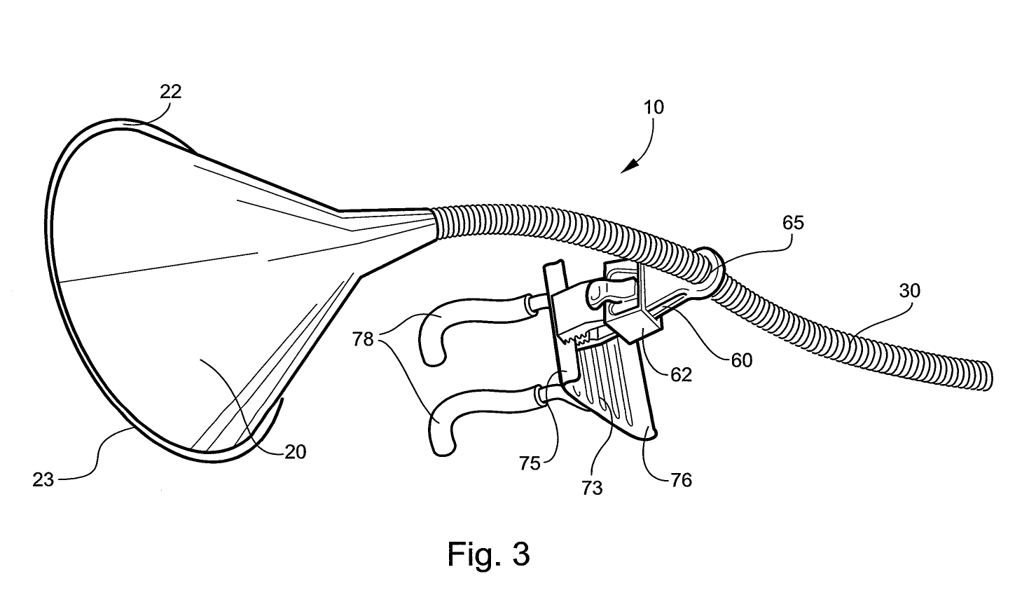

Funnel shaped device 20 (FIGS. 1-3), as used herein, describes a device which is essentially a pipe having a wide opening 22 at its proximal end 26 and a narrow opening 24 at its distal end 28. The wide opening 22 is defined by a rim 23 at the device's most proximal end. In the instant invention, the funnel shaped device 20 is used to channel liquid, namely urine, into a flexible conduit 30 with as little spillage as possible. In one embodiment of the present invention, the funnel shaped device 20 may be comprised of a material including, but not limited to, a plastic, a glass, a metal, a paper, or a combination thereof. In another embodiment of the present invention, the funnel shaped device 20 further includes a handle 25 which may emanate from the rim 23 of the device 20. In still another embodiment, a handle 25 may emanate from both the rim 25 and the outside of the device 20. In still another embodiment, the funnel shaped device 20 may be recyclable. In still another embodiment, the funnel shaped device 20 may be disposable.

Flexible conduit 30, as used herein, describes a device which is a hollow tube designed to carry fluids from one location to another. The flexible conduit 30 has a proximal end 36 and a distal end 38. The proximal end 36 of the conduit 30 is secured to the distal end 28 of the funnel shaped device 20 and the distal end 38 of the conduit 30 is directed into the bowl 44 of a toilet 40. In one embodiment of the instant invention, the flexible conduit 30 may be comprised of a material including, but not limited to, a plastic, a metal, a paper, a rubber, a silicone, or a combination thereof. In another embodiment of the present invention, the flexible conduit 30 is rigid enough to allow the funnel shaped device 20 of urinal aid 10 to extend vertically up from the toilet bowl and remain in position without any assistance from a person using the device (i.e., the device is hands free). In still another embodiment, the flexible conduit 30 is a plastic corrugated pipe. In yet another embodiment, the flexible conduit is a metal corrugated pipe. In one embodiment of the present invention, the flexible conduit 30 is permanently secured to the funnel shaped device 20. In another embodiment of the present invention, the flexible conduit 30 is releasably secured to the funnel shaped device 20.

Clamping means 60, as used herein, describes a device which is detachably engaged to the flexible conduit 30. Looking to the figures, the clamping means is comprised of a body 62, a guide hole 65, a first side 66 and a second side 68. In the embodiment illustrated in the Figures, the clamping means 60 functions by slideably engaging the flexible conduit 30 which passes through the guide hole 65. The guide hole 65 is narrow enough in relation to the flexible conduit 30 to retain the flexible conduit 30 in place after a user has placed it in a desired location. In one embodiment of the present invention, the clamping means may be comprised of a material including, but not limited to, plastic, metal, wood, or a combination thereof. In another embodiment of the present invention, the clamping means 60 may be a device similar to a spring clamp. In still another embodiment, the clamping means 60 is detachably engaged to a bowl hook 70.

Bowl hook 70, as used herein, describes a device which includes a body 72 with an inner surface 73 and an outer surface 74. Emanating from the body 72 is an upper lip 75, a lower lip 76 and one or more prongs 78. Looking to the Figures there is illustrated an embodiment wherein a pair of prongs 78 emanates from the body 72 of the bowl hook 70. In another embodiment, a plurality of prongs 78 ranging in width from 0.5 cm to 2.0 cm may emanate from the body 72 of the bowl hook 70. In still another embodiment, a plurality of prongs 78 ranging in width from 0.75 cm to 1.75 cm may emanate from the body 72 of the bowl hook 70. In yet another embodiment, a plurality of prongs 78 ranging in width from 1.0 cm to 1.5 cm may emanate from the body 72 of the bowl hook 70. In still another embodiment, a single prong 78 ranging in width from 1.0 to 8.0 cm may emanate from the body 72 of the bowl hook 70. In yet another embodiment, a single prong 78 ranging in width from 2.0 to 7.0 cm may emanate from the body 72 of the bowl hook 70. In still another embodiment, a single prong 78 ranging in width from 3.0 to 6.0 cm may emanate from the body 72 of the bowl hook 70.

In one embodiment of the present invention, the clamping means 60 and the bowl hook 70 are permanently coupled to one another. In another embodiment of the present invention, the clamping means 60 and the bowl hook 70 are a single device. In still another embodiment, a prong 78 further includes a gripping material coating 79 to the outside surface of at least part of the prong 78. The gripping material coating 79 serves to aid in retaining the bowl hook 70 in its desired position once it has been engaged to a toilet 40. The gripping material coating may include a material including, but not limited to, a rubber, a silicone, a plastic, or a combination thereof.

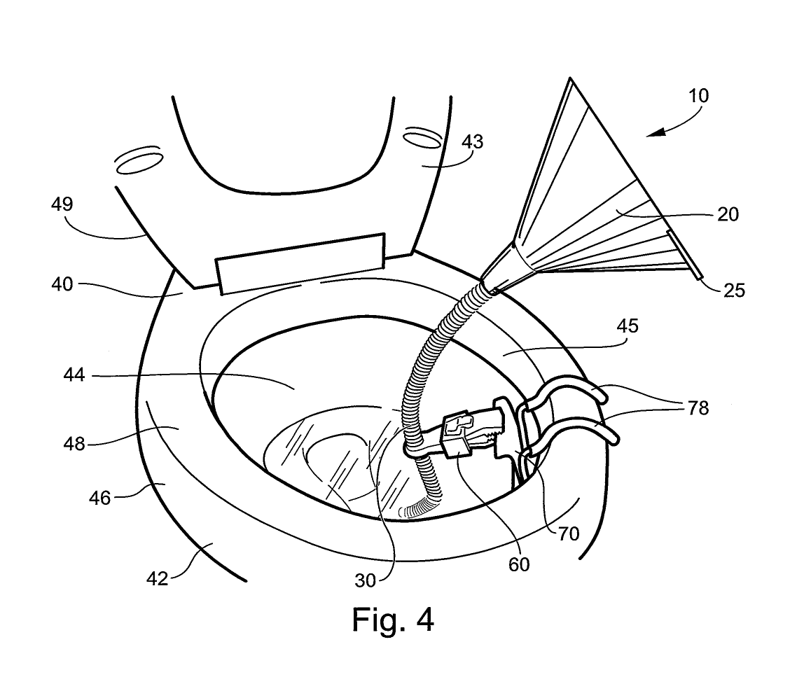

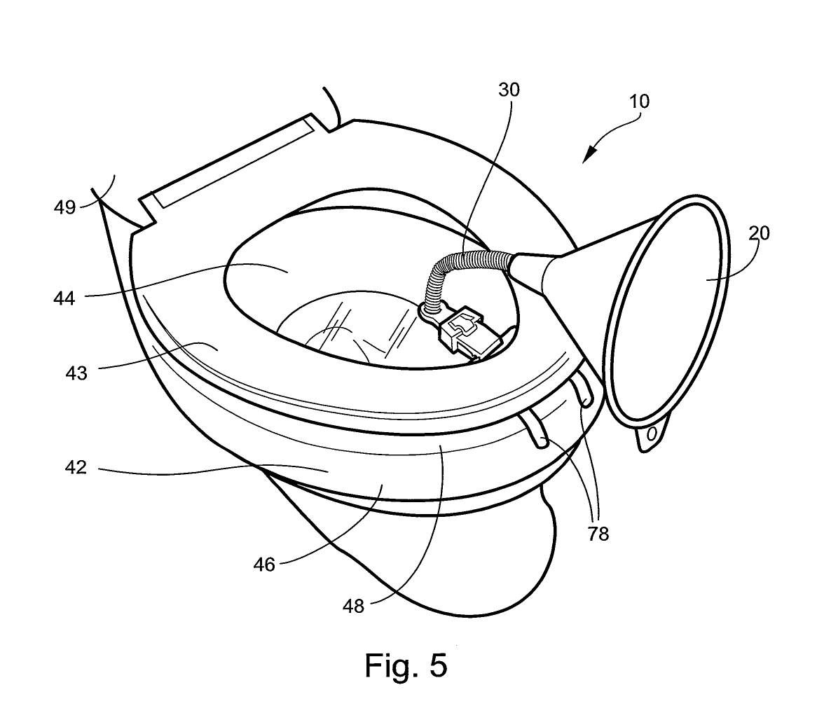

Toilet 40 and toilet bowl 44, as used herein, describes a typical toilet as known in the art. The toilet may be either a residential or commercial/industrial toilet. The toilet 40 may include a base 42 which is secured to the floor which emanates up to form the toilet bowl 44 which includes an inner bowl 45 and an outer bowl 46. FIGS. 4 and 5 illustrate that the toilet includes a rim 48 which is substantially parallel to the floor and a tank 49 which sits at the back of the base 42. A toilet seat 43 is generally secured to the base by a hinge which allows the seat 43 to be raised and lowered as desired.

Looking to FIGS. 4 and 5, there is illustrated an embodiment of the urinal aid 10 as it is engaged to a toilet 40. The urinal aid 10 is releasably mounted to the toilet 40 by placing the distal end of the flexible conduit 30 within the inner bowl 45 of the toilet 40 and placing the prongs 78 of the bowl hook 70 onto the rim 48 of the toilet. The inner surface 73 of the body 72 of the bowl hook 70 rests against a substantially vertical portion of the inner bowl 45 and the clamping means 60 extends away from the outer surface 74 of the body 72 of the bowl hook 70 toward the center of the inner bowl 45 of the toilet 40. The distal end 38 of the flexible conduit 30 extends down into the inner bowl into or near the water and the proximal end 36 extends upward and is engaged to the funnel shaped device 20. Looking to FIG. 5, the toilet seat 43 may be lowered down onto the prongs 78, resting on or above the rim 48 of the toilet 40 to aid in retaining the urinal aid 10 in place while it is in use.

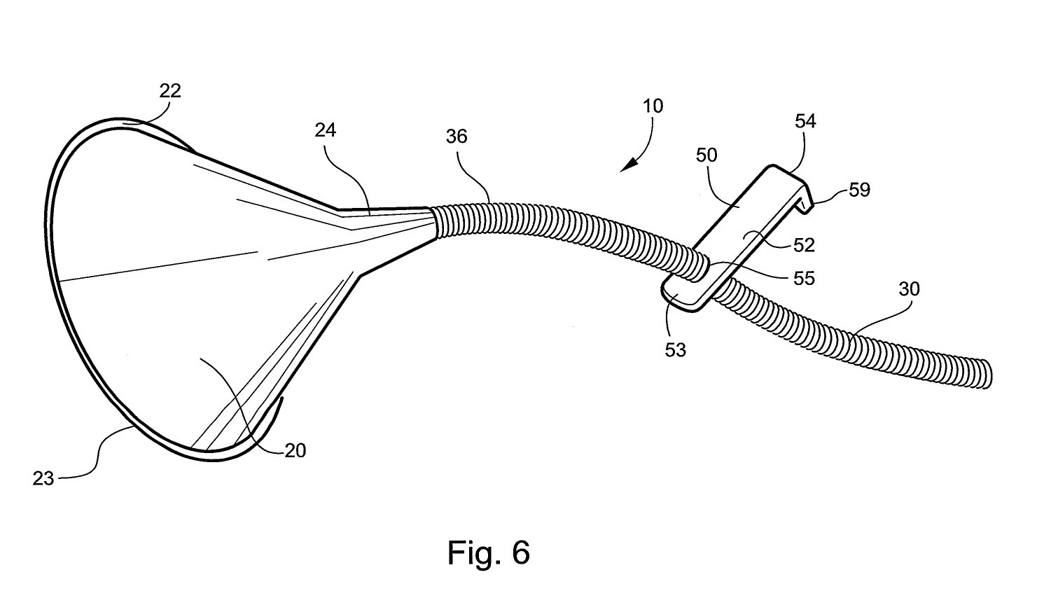

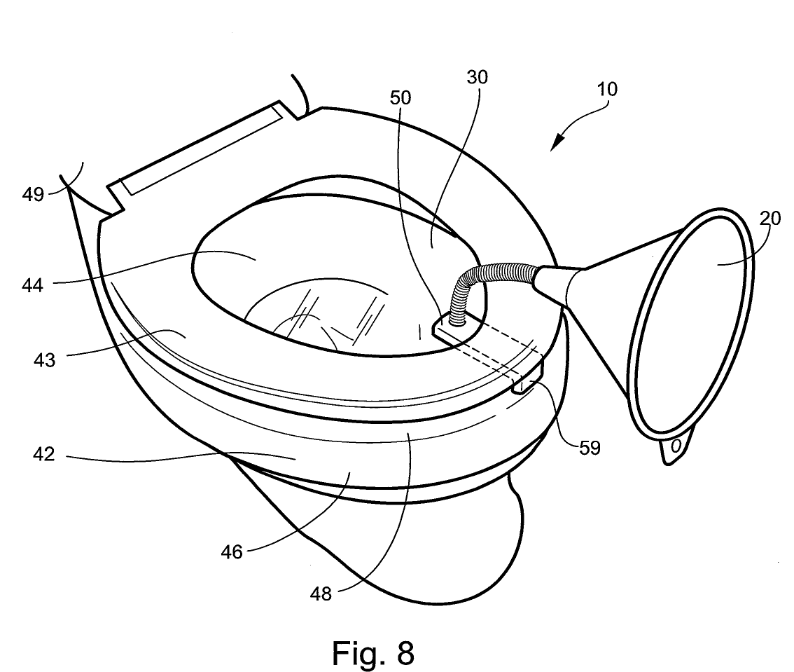

Looking now to FIGS. 6, 7 and 8, there is illustrated another embodiment of the present invention. FIG. 6 shows a urinal aid 10 comprising a funnel shaped device 20 having a wide opening 22 at a proximal end 26 and a narrow opening 24 at a distal end 28. The urinal aid 10 includes a flexible conduit 30 having a proximal end 36 and a distal end 38, where the conduit 30 is connected at its proximal end 36 to the distal end 28 of the funnel shaped device 20 and its distal end 38 directed toward a toilet bowl 44. The urinal aid 10 further includes an arm bracket 50 detachably engaged with the flexible conduit 30 where the arm bracket 50 is engaged between a toilet bowl 44 and a toilet seat 43 to secure the urinary aid 10 to the side of the toilet bowl 44 (i.e. the inner bowl 45) and hold the urinary aid 10 in place and allowing for the rapid detachment of the aid 10 from the toilet 40.

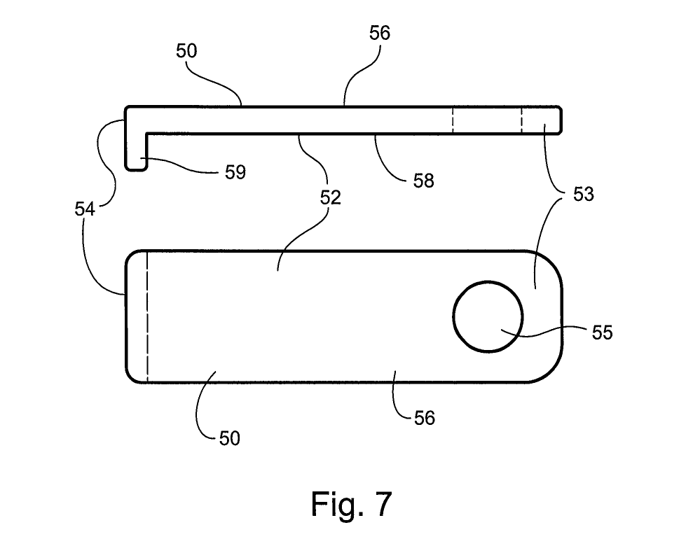

The arm bracket 50 includes a body 52, a proximal end 53 and a distal end 54. A guide hole 55 is located near the proximal end 53 of the arm bracket and a lip 59 is located on the distal end 54 of the arm bracket. The arm bracket 50 also includes a first side 56 and a second side 58 (FIG. 7). The lip 59 is integral to the device to ensure that the arm bracket 50 remains in place between the toilet seat 43 and the toilet bowl 44 while the urinal aid is being set up and/or in use. The lip 59 engages with the outer bowl 46 while the second side 58 engages with the rim 48 of the toilet, thus preventing the arm bracket 50 from sliding back into the bowl due to manipulation of the funnel shaped device and/or the flexible conduit. In the above embodiment, the funnel shaped device 20 may be comprised of a plastic, a glass, a metal, a paper, or a combination thereof. In the above embodiment, the flexible conduit 30 may be comprised of a plastic, a metal, a paper, a rubber, a silicone, or a combination thereof. In the above embodiment, the arm bracket may be comprised of a plastic, a metal, a paper, a rubber, a silicone, or a combination thereof. In the above embodiment, the funnel shaped device 20 may further include a handle 25. In still another embodiment, the arm bracket may further including a gripping material coating a part of its ends and/or body.

In another embodiment, a plurality of prongs ranging in width from 0.5 cm to 2.0 cm may emanate from the body 52 of the arm bracket 50. In still another embodiment, a plurality of prongs ranging in width from 0.75 cm to 1.75 cm may emanate from the body 52 of the arm bracket 50. In yet another embodiment, a plurality of prongs ranging in width from 1.0 cm to 1.5 cm may emanate from the body 52 of the arm bracket 50. In still another embodiment, a single prong ranging in width from 1.0 to 8.0 cm may emanate from the body 52 of the arm bracket 50. In yet another embodiment, a single prong ranging in width from 2.0 to 7.0 cm may emanate from the body 52 of the arm bracket 50. In still another embodiment, a single prong ranging in width from 3.0 to 6.0 cm may emanate from the body 52 of the arm bracket 50.

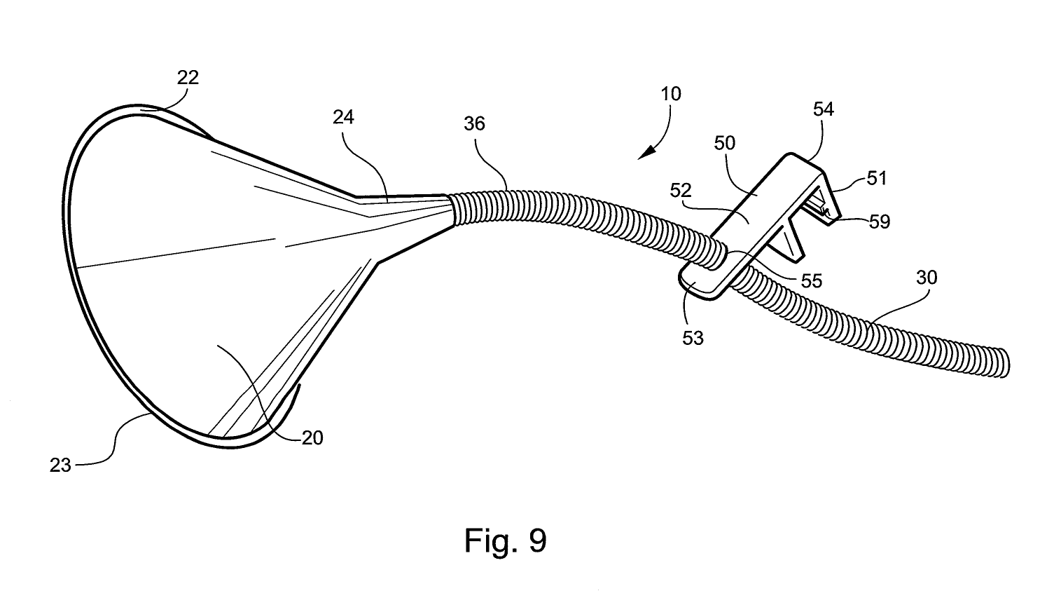

Looking now to FIGS. 9, 10 and 11, there is illustrated another embodiment of the present invention. FIG. 9 shows a urinal aid 10 comprising a funnel shaped device 20 having a wide opening 22 at a proximal end 26 and a narrow opening 24 at a distal end 28. The urinal aid 10 includes a flexible conduit 30 having a proximal end 36 and a distal end 38, where the conduit 30 is connected at its proximal end 36 to the distal end 28 of the funnel shaped device 20 and its distal end 38 directed toward a toilet bowl 44. The urinal aid 10 further includes an arm bracket 50 detachably engaged with the flexible conduit 30 where the arm bracket 50 is engaged to a toilet bowl 44 and to secure the urinary aid 10 to the rim of the toilet bowl 48 (i.e. the inner bowl 45 and the outer bowl 46) and hold the urinary aid 10 in place and allowing for the rapid detachment of the aid 10 from the toilet 40.

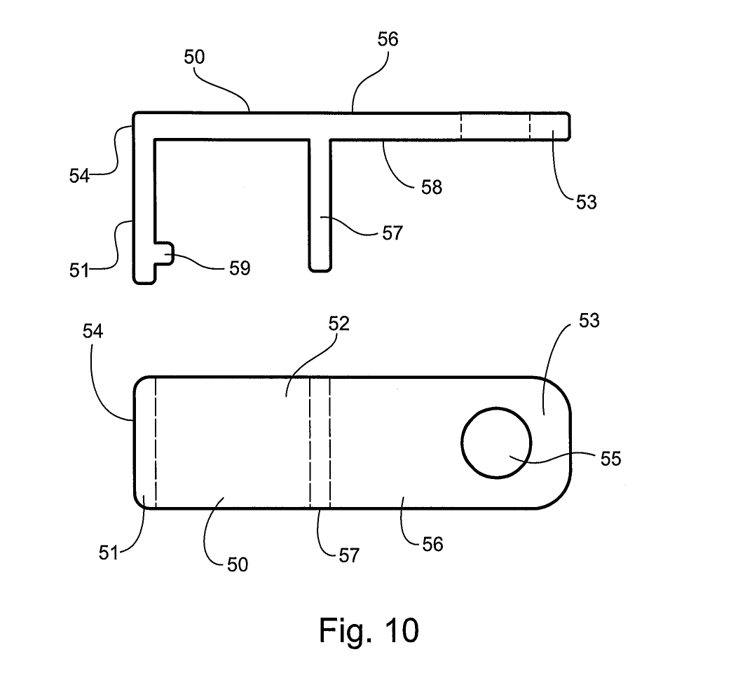

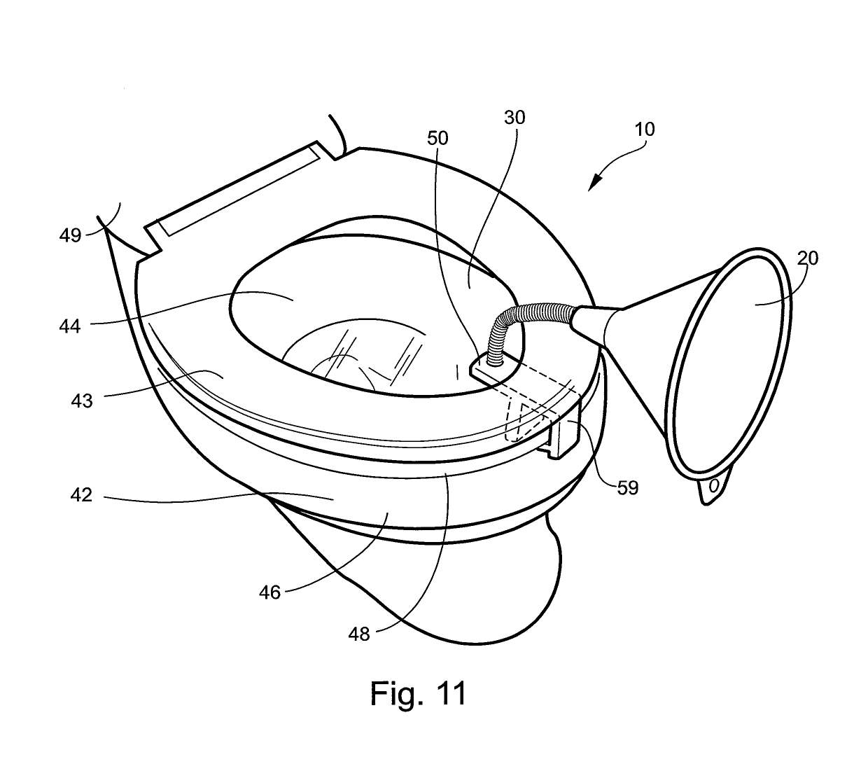

In the above embodiment illustrated in FIG. 10, the arm bracket 50 includes a body 52, a proximal end 53, a distal end 54 and a guide hole 55 located near the proximal end 53 of the arm bracket. A primary arm 51 emanates from the second side 58 of the distal end 54 of the arm bracket 50. The primary arm 51 may also include a lip 59 located on the distal end of the primary arm. A secondary arm 57 also emanates from the second side 58 of the arm bracket 50. The distance between the primary arm 51 and the secondary arm 57 is enough to allow the primary arm 51 and the secondary arm 57 to fit securely around the rim 48 of a toilet and remain in place as illustrated in FIG. 11. The arm bracket 50 also includes a first side 56 and a second side 58. The lip 59 is integral to the device to ensure that the arm bracket 50 remains in place engaging the inner bowl 45, outer bowl 46 and rim of the toilet in order to maintain the location of the urinal aid 10 in a hands-free manner. The primary arm 51 and the lip 59 engage the outer bowl 46 and the secondary arm 57 engages the inner bowl 45 while the second side 58 engages with the rim 48 of the toilet, thus preventing the arm bracket 50 from sliding back into the bowl or out of the toilet due to manipulation of the funnel shaped device and/or the flexible conduit. In the above embodiment, the funnel shaped device 20 may be comprised of a plastic, a glass, a metal, a paper, or a combination thereof. In the above embodiment, the flexible conduit 30 may be comprised of a plastic, a metal, a paper, a rubber, a silicone, or a combination thereof. In the above embodiment, the arm bracket may be comprised of a plastic, a metal, a paper, a rubber, a silicone, or a combination thereof. In the above embodiment, the funnel shaped device 20 may further include a handle 25. In still another embodiment, the arm bracket may further including a gripping material coating a part of its ends and/or body.

In one embodiment of the present invention, all of the parts of the urinal aid 10 may be disassembled from one another making the parts easily washable either in a dishwasher or by hand. In another embodiment of the present invention, all of the parts of the urinal aid 10 may be comprised of materials which are recyclable. In still another embodiment, all of the parts of the urinal aid 10 may be comprised of materials which are disposable and biodegradable.

The instant invention also includes a method for using a urinal aid which comprises the following steps: providing a urinal aid 10 comprised of a funnel shaped device 20 which has a wide opening 22 at its proximal end 26 and a narrow opening 24 at its distal end 28; a flexible conduit 30 which has a proximal end 36 and a distal end 38 where the proximal end 36 of the flexible conduit 30 is engaged to the distal end 28 of the funnel shaped device 20 and the distal end 38 of the flexible conduit is directed toward a toilet bowl 44; a toilet engagement device detachably engaged with the flexible conduit 30 where the toilet engagement device is selected from the group comprising: a clamping means 60 is detachably engaged with the flexible conduit 30 and a bowl hook 70 is detachably engaged to the clamping means 70 or an arm bracket 50 detachably engaged with the flexible conduit 30; securing the urinary aid 10 to a toilet bowl by lifting the seat 43 of the toilet 40; placing the distal end 38 of the flexible conduit 30 within the toilet bowl 44; engaging the toilet engagement device (i.e. bowl hook 70 or arm bracket 50) to a rim 48 and/or side of the toilet bowl 44 in order to hold the urinary aid 10 in place; urinating into the funnel shaped device 20; flushing the toilet 40 after completing urination; detaching the urinal aid 10 from the toilet 40; and lowering the seat 43 of the toilet back to its horizontal resting position.

The method described above may further include the step of lowering the seat 43 of the toilet 40 after engaging the toilet engagement device (i.e. the bowl hook 70 or the arm bracket 50) with the rim 48 and/or side of the toilet bowl 44 to aid in holding the urinary aid 10 in place while it is used. In one embodiment of the above method, the funnel shaped device 20 may be comprised of a material including, but not limited to, a plastic, a glass, a metal, a paper, or a combination thereof. In another embodiment of the above method, the funnel shaped device 20 further includes a handle 25 which may emanate from the rim 23 of the device 20. In still another embodiment, a handle 25 may emanate from both the rim 25 and the outside of the device 20. In still another embodiment, the funnel shaped device 20 may be recyclable. In still another embodiment, the funnel shaped device 20 may be disposable.

In one embodiment of the above method, the flexible conduit 30 may be comprised of a material including, but not limited to, a plastic, a metal, a paper, a rubber, a silicone, or a combination thereof. In another embodiment of the above method, the flexible conduit 30 is rigid enough to allow the funnel shaped device 20 of urinal aid 10 to extend vertically up from the toilet bowl and remain in position without any assistance from a person using the device (i.e., the device is hands free). In still another embodiment, the flexible conduit 30 is a plastic corrugated pipe. In yet another embodiment, the flexible conduit is a metal corrugated pipe. In one embodiment of the above method, the flexible conduit 30 is permanently secured to the funnel shaped device 20. In another embodiment of the above method, the flexible conduit 30 is releasably secured to the funnel shaped device 20.

In one embodiment of the above method, the clamping means 60 may be comprised of a material including, but not limited to, plastic, metal, wood, or a combination thereof. In another embodiment of the above method, the clamping means 60 may be a device similar to a spring clamp. In still another embodiment, the clamping means 60 is detachably engaged to a bowl hook 70. In another embodiment, a plurality of prongs 78 ranging in width from 0.5 cm to 2.0 cm may emanate from the body 72 of the bowl hook 70. In still another embodiment, a plurality of prongs 78 ranging in width from 0.75 cm to 1.75 cm may emanate from the body 72 of the bowl hook 70. In yet another embodiment, a plurality of prongs 78 ranging in width from 1.0 cm to 1.5 cm may emanate from the body 72 of the bowl hook 70. In still another embodiment, a single prong 78 ranging in width from 1.0 to 8.0 cm may emanate from the body 72 of the bowl hook 70. In yet another embodiment, a single prong 78 ranging in width from 2.0 to 7.0 cm may emanate from the body 72 of the bowl hook 70. In still another embodiment, a single prong 78 ranging in width from 3.0 to 6.0 cm may emanate from the body 72 of the bowl hook 70.

In one embodiment of the above method, the clamping means 60 and the bowl hook 70 are permanently coupled to one another. In another embodiment of the above method, the clamping means 60 and the bowl hook 70 are a single device. In still another embodiment, a prong 78 further includes a gripping material coating 79 to the outside surface of at least part of the prong 78. The gripping material coating 79 serves to aid in retaining the bowl hook 70 in its desired position once it has been engaged to a toilet 40. The gripping material coating may include a material including, but not limited to, a rubber, a silicone, a plastic, or a combination thereof.

In one embodiment of the above method, an arm bracket 50 as described above and illustrated in FIGS. 6, 7 and 8 may be used. In another embodiment of the above method, an arm bracket 50 as described above and illustrated in FIGS. 9, 10 and 11 may be used. In still another embodiment, an arm bracket 50 as described above and illustrated in FIGS. 6-11 may be used.

The instant invention also includes a method for using a urinal aid which comprises the following steps: providing a urinal aid 10 comprised of a funnel shaped device 20 which has a wide opening 22 at its proximal end 26 and a narrow opening 24 at its distal end 28; a flexible conduit 30 which has a proximal end 36 and a distal end 38, where the conduit 30 is connected at its proximal end 36 to the distal end 28 of the funnel shaped device 20 and its distal end 38 directed toward a toilet bowl 44. The urinal aid 10 further includes an arm bracket 50 detachably engaged with the flexible conduit 30 securing the urinary aid 10 to a toilet bowl by lifting the seat 43 of the toilet 40; placing the distal end 38 of the flexible conduit 30 within the toilet bowl 44; engaging the arm bracket 50 to a rim 48 or side of the toilet bowl 44 with the lip 59 protruding toward or engaging the outer bowl 46 of the toilet (to prevent the arm bracket from disengaging from the toilet bowl), lowering the seat 43 of the toilet over the arm bracket 50 in order to hold the urinary aid 10 in place; urinating into the funnel shaped device 20; flushing the toilet 40 after completing urination; raising the seat 43 followed by detaching the urinal aid 10 from the toilet 40; and lowering the seat 43 of the toilet back to its horizontal resting position.

In one embodiment of the above method, the funnel shaped device 20 may be comprised of a material including, but not limited to, a plastic, a glass, a metal, a paper, or a combination thereof. In another embodiment of the above method, the funnel shaped device 20 further includes a handle 25 which may emanate from the rim 23 of the device 20. In still another embodiment, a handle 25 may emanate from both the rim 25 and the outside of the device 20. In still another embodiment, the funnel shaped device 20 may be recyclable. In still another embodiment, the funnel shaped device 20 may be disposable.

In one embodiment of the above method, the flexible conduit 30 may be comprised of a material including, but not limited to, a plastic, a metal, a paper, a rubber, a silicone, or a combination thereof. In another embodiment of the above method, the flexible conduit 30 is rigid enough to allow the funnel shaped device 20 of urinal aid 10 to extend vertically up from the toilet bowl and remain in position without any assistance from a person using the device (i.e., the device is hands free). In still another embodiment, the flexible conduit 30 is a plastic corrugated pipe. In yet another embodiment, the flexible conduit is a metal corrugated pipe. In one embodiment of the above method, the flexible conduit 30 is permanently secured to the funnel shaped device 20. In another embodiment of the above method, the flexible conduit 30 is releasably secured to the funnel shaped device 20.

In one embodiment of the above method, the arm bracket 50 may include a body 52, a proximal end 53 and a distal end 54. A guide hole 55 is located near the proximal end 53 of the arm bracket and a lip 59 is located on the distal end 54 of the arm bracket. The arm bracket 50 also includes a first side 56 and a second side 58. In another embodiment, the arm bracket may be comprised of a plastic, a metal, a paper, a rubber, a silicone, or a combination thereof. In still another embodiment, the arm bracket may further including a gripping material coating a part of its ends and/or body.

In another embodiment, a plurality of prongs ranging in width from 0.5 cm to 2.0 cm may emanate from the body 52 of the arm bracket 50. In still another embodiment, a plurality of prongs ranging in width from 0.75 cm to 1.75 cm may emanate from the body 52 of the arm bracket 50. In yet another embodiment, a plurality of prongs ranging in width from 1.0 cm to 1.5 cm may emanate from the body 52 of the arm bracket 50. In still another embodiment, a single prong ranging in width from 1.0 to 8.0 cm may emanate from the body 52 of the arm bracket 50. In yet another embodiment, a single prong ranging in width from 2.0 to 7.0 cm may emanate from the body 52 of the arm bracket 50. In still another embodiment, a single prong ranging in width from 3.0 to 6.0 cm may emanate from the body 52 of the arm bracket 50.

The instant invention also includes a method for using a urinal aid which comprises the following steps: providing a urinal aid 10 comprised of a funnel shaped device 20 which has a wide opening 22 at its proximal end 26 and a narrow opening 24 at its distal end 28; a flexible conduit 30 which has a proximal end 36 and a distal end 38, where the conduit 30 is connected at its proximal end 36 to the distal end 28 of the funnel shaped device 20 and its distal end 38 directed toward a toilet bowl 44. The urinal aid 10 further includes an arm bracket 50 detachably engaged with the flexible conduit 30 securing the urinal aid 10 to a toilet bowl by lifting the seat 43 of the toilet 40, placing the distal end 38 of the flexible conduit 30 within the toilet bowl 44, engaging the arm bracket 50 to the rim 48, inner bowl 45 and outer bowl 46 by spreading the primary arm 51 and secondary arm 57 apart and placing them over the rim 48 with the lip 59 engaging the outer bowl 46 of the toilet (to prevent the arm bracket from disengaging from the toilet bowl), urinating into the funnel shaped device 20, flushing the toilet 40 after completing urination, detaching the urinal aid 10 from the toilet 40 and lowering the seat 43 of the toilet back to its horizontal resting position or leaving the urinal aid in its current position for use again later.

In one embodiment of the above method, the funnel shaped device 20 may be comprised of a material including, but not limited to, a plastic, a glass, a metal, a paper, or a combination thereof. In another embodiment of the above method, the funnel shaped device 20 further includes a handle 25 which may emanate from the rim 23 of the device 20. In still another embodiment, a handle 25 may emanate from both the rim 25 and the outside of the device 20. In still another embodiment, the funnel shaped device 20 may be recyclable. In still another embodiment, the funnel shaped device 20 may be disposable.

In one embodiment of the above method, the flexible conduit 30 may be comprised of a material including, but not limited to, a plastic, a metal, a paper, a rubber, a silicone, or a combination thereof. In another embodiment of the above method, the flexible conduit 30 is rigid enough to allow the funnel shaped device 20 of urinal aid 10 to extend vertically up from the toilet bowl and remain in position without any assistance from a person using the device (i.e., the device is hands free). In still another embodiment, the flexible conduit 30 is a plastic corrugated pipe. In yet another embodiment, the flexible conduit is a metal corrugated pipe. In one embodiment of the above method, the flexible conduit 30 is permanently secured to the funnel shaped device 20. In another embodiment of the above method, the flexible conduit 30 is releasably secured to the funnel shaped device 20.

In one embodiment of the above method, the arm bracket 50 includes a body 52, a proximal end 53, a distal end 54 and a guide hole 55 located near the proximal end 53 of the arm bracket. A primary arm 51 emanates from the second side 58 of the distal end 54 of the arm bracket 50. The primary arm 51 may also include a lip 59 located on the distal end of the primary arm. A secondary arm 57 also emanates from the second side 58 of the arm bracket 50. The distance between the primary arm 51 and the secondary arm 57 is enough to allow the primary arm 51 and the secondary arm 57 to fit securely around the rim 48 of a toilet and remain in place as illustrated in FIG. 11. The arm bracket 50 also includes a first side 56 and a second side 58. The lip 59 is integral to the device to ensure that the arm bracket 50 remains in place engaging the inner bowl 45, outer bowl 46 and rim of the toilet in order to maintain the location of the urinal aid 10 in a hands-free manner. The primary arm 51 and the lip 59 engage the outer bowl 46 and the secondary arm 57 engages the inner bowl 45 while the second side 58 engages with the rim 48 of the toilet, thus preventing the arm bracket 50 from sliding back into the bowl or out of the toilet due to manipulation of the funnel shaped device and/or the flexible conduit. In another embodiment, the arm bracket 50 may be comprised of a plastic, a metal, a paper, a rubber, a silicone, or a combination thereof.

In still another embodiment, the arm bracket 50 may further including a gripping material coating a part of its ends and/or body.

The present invention may be embodied in other forms without departing from the spirit and the essential attributes thereof, and, accordingly, reference should be made to the appended claims, rather than to the foregoing specification, as indicating the scope of the invention. The invention illustratively discloses herein suitably may be practiced in the absence of any element which is not specifically disclosed herein.

What is claimed is:

1. A urinal aid comprising:
a funnel shaped device having a wide opening at a proximal end and a narrow opening at a distal end;
a flexible conduit having a proximal end and a distal end, said conduit being connected at its proximal end to the distal end of said funnel shaped device and having its distal end directed toward a toilet bowl;
a toilet engagement device detachably engaged with said flexible conduit, said toilet engagement device being selected from the group comprising:
a clamping means detachably engaged with said flexible conduit and a bowl hook detachably engaged to said clamping means wherein said hook is engaged to a toilet bowl to secure the urinary aid to the side of the toilet bowl and hold the urinary aid in place and may be rapidly detached from the toilet bowl; or
an arm bracket detachably engaged with said flexible conduit wherein said arm bracket is engaged to a toilet bowl to secure the urinary aid to the side of the toilet bowl and hold the urinary aid in place and may be rapidly detached from the toilet bowl.

2. The urinal aid of claim 1 wherein said funnel shaped device being comprised of a plastic, a glass, a metal, a paper, or a combination thereof.

3. The urinal aid of claim 2 wherein said funnel shaped device being disposable.

4. The urinal aid of claim 1 wherein said funnel shaped device further including a handle.

5. The urinal aid of claim 1 wherein said flexible conduit being comprised of a plastic, a metal, a paper, a rubber, a silicone, or a combination thereof.

6. The urinal aid of claim 1 wherein said bowl hook including one or more prongs emanating from a body.

7. The urinal aid of claim 6 wherein said prongs further including a gripping material coating a part of said prongs.

8. A method of using a urinal aid comprising the steps of:
providing said urinal aid comprising:
a funnel shaped device having a wide opening at a proximal end and a narrow opening at a distal end;
a flexible conduit having a proximal end and a distal end, said conduit being connected at its proximal end to the distal end of said funnel shaped device and having its distal end directed toward a toilet bowl;
a toilet engagement device detachably engaged with said flexible conduit, said toilet engagement device being selected from the group comprising:
a clamping means detachably engaged with the flexible conduit and a bowl hook detachably engaged to said clamping means; or
an arm bracket detachably engaged with said flexible conduit;
securing said urinary aid to a toilet bowl by:
lifting the seat of the toilet;
placing the distal end of said flexible conduit within said toilet bowl;
engaging said toilet engagement device to a rim or side of said toilet bowl in order to hold the urinary aid in place;
urinating into said funnel shaped device;
flushing said toilet after completing urination;
detaching said urinal aid from said toilet bowl; and
lower the seat of the toilet.

9. The method of claim 8 further including the step of lowering the seat of the toilet after engaging the toilet engagement device with the rim and/or side of the toilet bowl to aid in holding the urinary aid in place while it is used.

10. The method of claim 8 wherein said funnel shaped device being comprised of a plastic, a glass, a metal, a paper, or a combination thereof.

11. The method of claim 10 wherein said funnel shaped device being disposable.

12. The method of claim 8 wherein said funnel shaped device further including a handle.

13. The method of claim 8 wherein said flexible conduit being comprised of a plastic, a metal, a paper, a rubber, a silicone, or a combination thereof.

14. The method of claim 8 wherein said bowl hook including one or more prongs emanating from a body.

15. The method of claim 14 wherein said prongs further including a gripping material coating a part of said prongs.

16. A urinal aid comprising:
a funnel shaped device having a wide opening at a proximal end and a narrow opening at a distal end;
a flexible conduit having a proximal end and a distal end, said conduit being connected at its proximal end to the distal end of said funnel shaped device and having its distal end directed toward a toilet bowl;
an arm bracket detachably engaged with the flexible conduit wherein said arm bracket is engaged between a toilet bowl and a toilet seat to secure the urinary aid to the side of the toilet bowl and hold the urinary aid in place and may be rapidly detached from the toilet bowl.

17. The urinal aid of claim 16 wherein said funnel shaped device being comprised of a plastic, a glass, a metal, a paper, or a combination thereof;
wherein said flexible conduit being comprised of a plastic, a metal, a paper, a rubber, a silicone, or a combination thereof; and
wherein said arm bracket being comprised of a plastic, a metal, a paper, a rubber, a silicone, or a combination thereof.

18. The urinal aid of claim 18 wherein said funnel shaped device further including a handle.
